(19) United States
(12) Patent Application Publication
(10) Pub. No.: US 2003/0114534 A1
(43) Pub. Date: Jun. 19, 2003

(54) Pharmaceutical preparation for apthous ulcers

(76) Inventor: Peter Arsenault, Dracut, MA (US)

Correspondence Address: Joseph E. Funk, PO Box 661, Londonderry, NH, 03053, US

(21) Appl. No.: 10/022,638

(22) Filed: Dec. 17, 2001

(51) Int. Cl. A61K 31/137; A61K 31/045

(52) U.S. Cl. 514/649; 514/724

(57) ABSTRACT

The present invention relates to a pharmaceutical preparation for treating apthous ulcers in the human mouth, by rinsing vigorously with a pharmaceutical rinse having a composition comprising ethyl alcohol in a concentration in the order of 50% weight per unit volume and lidocaine in a concentration in the order of 1.0-2.0% weight per unit volume at a periodicity of at least 3 to 4 times daily for thirty seconds each time, followed by expectoration; or by applying the preparation in the form of a lozenge or gel directly to the apthous ulcers.

### FIELD OF THE INVENTION

The present invention relates to a new pharmaceutical preparation for the treatment of apthous ulcers.

### BACKGROUND OF THE INVENTION

An apthous ulcer is a painful oral ulcer, which exists in connection with a disease known as apthous stomatitis and is one of the most common soft tissue diseases seen by dentists. Apthous ulcers occur in the oral cavity in small groups or singularly. Although there has been a great deal of research on the problem of these ulcers, their cause is essentially unknown, and truly effective treatment for curing same and/or relieving the discomfort of same has not been established until conception of the present invention.

An apthous ulcer is a recurrent, painful superficial oral ulcer that persists from 10 to 14 days. Apthous ulcers generally affect about 30% of the population. The lesions associated with apthous ulcers are known technically as recurrent apthous stomotitis (RAS) or apthous ulcers. RAS may be “minor” or of a more serious “major” variant.

Many etiologic factors have been suggested as causing apthous ulcerations, including allergic, microbial, diet, trauma, stress, endocrine, hereditary, by a fungus, or after enteric use of broad spectrum antibiotics. Heredity has been suggested as one; and although there is no evidence of genetic origin in the disease, apthous lesions have been frequently observed in several members of the same family.

There also appears to be an immune response against this organism as well as to the epithelial tissue it infects. The ensuing inflammatory response causes localized damage to the host in the form of degeneration and necrosis.

Protozoa has sometimes been suggested as a cause of this disease, but no conclusive evidence has ever been established that this is so.

While it is strongly suspected that trauma may contribute to the development of the apthous lesion, it is not considered to be the sole cause. Most likely the tissue was sensitized and made susceptible to the apthous lesion by a traumatic injury. However, it has been observed that a traumatic injury does not result in apthous ulcers in many cases, and particularly in connection with those people not prone to develop such lesions. Another observation is that the eruption of apthous lesions is frequently associated with the onset of menstruation and has also been correlated with post-menopausal women. It has further been observed that recurrent apthae completely subside during pregnancy in otherwise highly susceptible women. Regardless of this apparent hormonal relationship, there is no definite explanation of the mechanism of this phenomenon. Add to this the fact that apthae are frequently seen in men, and the roll of hormones in this disease becomes even more suspect.

There have been numerous reports in the literature of apthous outbreaks following the ingestion of certain drugs or foods. There is, however, little evidence that these eruptions are due to hypersensitive reactions; although some observers feel that hypersensitivity helps to support the reaction. It has also been noted that with apthous ulcers, as with many other kinds of ulcers, acute psychological factors appear to precipitate attacks of the disease. Although such psychological factors are difficult to analyze, it nevertheless has been popularly concluded that mental stress and psychological disturbances act as a precipitating mechanism to the disease, although they cannot be considered as the actual cause.

Apthous ulcers may affect patients of any age, but generally, those individuals between the ages of twenty and fifty years, and especially women, are prone to outbreaks. In general, apthous ulcers occur in women about twice as frequently as they occur in men. The frequency and severity of outbreaks vary remarkably between patients and within the same patient. In certain individuals, outbreaks occur once or twice a year, while in others, outbreaks may occur once or twice a month.

The outbreak of apthous ulcer lesions is generally preceded by a prodromal period of several days during which period the patient experiences a burning or itching sensation in the oral mucosal area. The onset may also be preceded by a low grade fever and/or lymph node irritation. Upon onset, well defined ulcers form of approximately two to ten millimeters in diameter. These ulcers are generally surrounded by an intense erythematous halo with the base of the ulcer covered by a grayish necrotic tissue. Large, long standing ulcers are called major aphthae and involve the mucous glands, muscle and connective tissue. At this level, the disease may be quite painful and heal with scarring. In certain individuals, the ulcers never really disappear, with a new ulcer appearing as the older one heals.

Turning now to the clinical appearance of apthous ulcers, they are characterized by small white spots associated with small ulcerations on the mucous membrane of the mouth. It has been noted that they are single, multiple, round or oval ulcerations. Apthous ulcers are extremely painful lesions. They first appear as small macular red lesions. These areas quickly undergo necrosis, leaving a sharply defined, rounded ulcer, usually varying from 2 to 5 mm. in diameter. The ulceration is fairly deep, with a yellow-white base representing necrotic tissue at the surface. The margins of the ulcer are somewhat indurate, and the marginal mucosa has a surrounding erythamatous zone. The marginal erythema ranges from slight to extensive, depending upon the degree of secondary bacterial involvement. The ulcer is present for approximately seven days, and it undergoes gradual healing. It heals, as a general rule, in approximately ten to fourteen days, and usually leaves no scarring.

Apthous ulcers are characterized by small white spots associated with small ulcerations on the mucous membrane of the mouth. Characteristically, there is a recurrent pattern of one or more of these ulcers. The lesions may reoccur as often as one month apart; and there are cases where for a period of years the individual is never without apthous lesions, new ones forming as the previous ones heal. In other cases, apthous ulcer attacks may occur two to three times during a year. The lesions often appear following some intense emotional stress, but they may first appear following a gradual change in environment or following an emotional situation, such as the early adjustment period of marriage, boarding school, new employment in a non-familiar environment, etc. Apthous ulcers have been found to occur in cyclic patterns in females. They may appear several days prior to the menstrual period. The first encounter with apthous stomatitis may follow the onset of menstruation. Women susceptible to this lesion often report freedom from the lesions during pregnancy. There is a tendency for a greater frequency of these lesions in females than in males; and although apthae occur at any age level, they seem to occur more often in young adults. The term “periadenitis mucosa necrotica recurrens” is sometimes used to describe large apthae that coalesce to form an elongated, deep ulcerated area.

From a symptomatic standpoint, it has been found that approximately 24 to 48 hours prior to the onset of an apthous lesion there is a vague discomfort, sometimes described as a tingling sensation, in the area. As the tissues undergo necrosis and an ulcer forms, the lesion becomes very painful. The apthous lesions are often considered to be the most painful of oral ulcerative lesions. The discomfort may become particularly intense during periods of fatigue. Turning to the histopathology of the disease, it has been found that the microscopic picture thereof is non-specific, generally showing an ulceration of the mucosa. The surface epithelium exhibits a central area of destruction. The connective tissue is densely infiltrated with lymphocytes, polymorphonuclear leukocytes, plasma cells, and histocytes. There is evidence of active fibrosis at the base and sides of the ulcerated area.

Differential diagnosis may include traumatic ulcer, acute herpetic stomatitis, stomatitis medicamentosa, and erythema multiforme. The diagnosis of apthous stomatitis is based upon the clinical manifestations and the patient's history. Biopsis are usually unnecessary and, due to the extreme discomfort involved, are avoided unless necessary to rule out other lesions considered in the differential diagnosis.

Many different substances and agents have heretofore been used in an attempt to cure and/or relieve the discomfort of apthous lesions. For example, cauterizing drugs (escharotic agents), such as phenol, chromic acid, alum and silver nitrate, have been used for many years. These agents alleviate pain by destruction of the small nerve endings. The healing time of the lesion is prolonged due to the escharotic action of these drugs on the surface epithelium and the active fibrosis at the base and sides of the ulcerated areas. Different vitamins have been tried, with inconsistent results; and various antibiotics have also been used, with conflicting results. One observer found that Aureomycin applied locally (250 mg. in 10 ml. of water) three times a day appeared to have a definite effect. The duration of the ulcers was reduced from approximately ten to five days, and there was an analgesic effect lasting one-half to two hours. Temporary relief has also been sought, and sometimes achieved, by using milk of magnesia, or various heavy syrups. The transient nature of their benefits renders these preparations impractical. More recently, mucous membrane adhering compounds (“Orabase”, Squibb trademark) have become available to eliminate irritants that delay healing. Other more exotic remedies have been tried with little or no success, such as vaccination with cowpox virus, lactobacillus containing materials, and nutrient supplements.

Corticosteroid agents have also been used in several ways for the treatment of apthous stomatitis. These agents possess anti-inflammatory properties and have been successful in suppressing or reducing inflammatory processes in the skin and mucous membranes. It has been found that topical applications directly to the apthous lesions avoid the systemic effect of corticosteroid. Topical applications are therefore preferred to the systemic route, especially if long-term treatment is anticipated, since even though some absorption occurs with topical steroids, it is never enough to be of real concern. Topical cortisones used for apthous lesions include hydrocortisone, prednisolone, and dexamethasone. These agents must be frequently and thoroughly applied to the ulcers in ointment or cream form. Corticosteroids, such as triamcinolone, have been incorporated into a base, such as “Orabase”, which has the property of adhering to mucous membrane. It is thought, however, that very little, if any, of the active medication is released from the heavy paste so as to be effective.

Attempts have been made to use intralesional injection to treat apthous lesions, this method concentrating the drug at the lesion, while usually avoiding significant systemic effects. The problem with this treatment, however, is that significant discomfort may be present, especially if the apthous lesions are numerous. In addition, patients with gastric ulcers may absorb enough steroids to complicate and aggravate their gastric ulcer.

In situations where patients have numerous lesions and, as a result, topical therapy is unsatisfactory due to the number of ulcers and their inaccessibility, attempts have been made to use systemic treatment. This form of treatment may result in severe complications, particularly with ulcer patients. It should only be used in very severe cases when it is essential that the patient not be incapacitated. Daily dosage of injections of corticosteroids must be gradually reduced. It is hazardous to suddenly stop systemic cortisone administrations, and this treatment should be avoided entirely where gastric ulcers are present.

In addition, in the acute form, several other treatments are presently available including: tetracycline mouth rinses, topical cortisone and anesthetic mouth rinses. Systemic analgesics also may be administered.

However, all of these therapies are fairly extreme and are generally reserved for multiple ulcers or major aphthae. All of these therapies should be carefully monitored.

It will therefore be seen that none of these prior approaches have proven altogether successful in the treatment of apthous ulcers, since none of them quickly cure the condition, and since many of them create other complications.

It is therefore a primary objective of this invention to provide a quick, convenient, safe and relatively painless method of treating apthous ulcers.

### SUMMARY AND DETAILED DESCRIPTION OF THE INVENTION

Applicant has discovered that a rinse solution comprising ethyl alcohol, typically in a 40% to 50% solution, although other concentrations may be used, and all concentrations hereinafter being referred to as “ethyl alcohol”) is an effective treatment for use in treating apthous ulcers when applied directly to the lesions by rinsing for 20 to 30 seconds at least 3 to 4 times daily. Although applicant believes the ethyl alcohol to be effective without the addition of other agents to treat the apthous ulcers, applicant has discovered that the addition of topical anesthetic such as lidocaine or benzocaine aids in numbing lesions. In addition, a flavoring added to the rinse, such as with mouthwash, is helpful.

Lidocaine (2-diethylaminoacetyl-2,6-xylidide) is known for its properties as a topical local anesthetic and serves to reduce the pain associated with apthous ulcers, alleviate the side effects of the rinse, while the ethyl alcohol acts to cause the lesions to heal or go into remission. The lidocaine or a pharmaceutically acceptable salt thereof is preferred in a concentration of 0.5 to 10% weight per unit volume, and preferably about 1-2% weight to per unit volume.

The applicant has found that the diligent use of the novel apthous ulcer rinse decreases the healing period of apthous ulcers from their normal period of 10 to 14 days down to 3 to 4 days.

Although many formulations are possible of the pharmaceutical composition contemplated by the applicant, four preferred formulations are set forth below. All formulations are for 100 ml of treatment composition, and the weight per unit volume of each element thereof is as follows:

| | |
|---|---|
| Formulation 1 | |
| Ethyl Alcohol | 40.0% |
| Lidocaine | 1.0% |
| Water | 59.0% |
| Flavoring & coloring | |
| Formulation 2 | |
| Ethyl Alcohol | 50.0% |
| Lidocaine | 2.0% |
| Water | 48.0% |
| Flavoring & coloring | |
| Formulation 3 | |
| Ethyl Alcohol | 45.0% |
| Lidocaine | 1.5% |
| Water | 53.5% |
| Flavoring & coloring | |
| Formulation 4 | |
| Ethyl Alcohol | 50.0% |
| Water | 50.0% |
| Flavoring & coloring | |

Normal use of the pharmaceutical rinse should begin at the first signs of an apthous ulcer and continue at the rate of at least 3 to 4 applications per day during waking hours, rinsing vigorously for thirty seconds each time, then expectorate. Although therapy should begin as early as possible, applicant has found that treatment of mature ulcers results in faster healing and thus believes that the rinse is effective when used at any stage of ulcer development.

Applicant has treated a number of human subjects with the pharmaceutical formulation and had surprisingly successful results in all the cases. In the study, the patients were initially seen at varying times after the appearance of ulcers and were advised to rinse vigorously for 30 second periods 3 to 4 times a day and expectorate. The results of the human case studies indicate that the pharmaceutical formulation, applied to apthous ulcers as a rinse, is able to successfully shorten the life of the ulcers. Representative case studies are set forth below:

Human Case Study 1

A 33 year old woman started treatment with a 40% ethyl alcohol formulation at the first sign of an apthous ulcer. The formulation was applied by rinsing for 30 second periods, four times a day and resulted in a complete remission of the ulcer in 1 day. Previously, she had frequent onsets of painful, large, apthous ulcers.

Human Case Study 2

A 38 year old man started treatment with a 40% ethyl alcohol formulation after mature apthous ulcers had developed. The formulation was applied by rinsing for 30 second periods, three times a day and resulted in a complete remission of the ulcer within 3 days after commencing use of the rinse.

Human Case Study 3

A 69 year old woman started treatment with a 40% ethyl alcohol formulation after mature apthous ulcers had developed. The formulation was applied by rinsing for 30 second periods, three times a day and resulted in a complete remission of the ulcer within 3 days after commencing use of the rinse.

Human Case Study 4

A 53 year old woman started treatment with a 40% ethyl alcohol formulation after mature apthous ulcers had developed. The formulation was applied by rinsing for 30 second periods, three times a day and resulted in a complete remission of the ulcer within 4 days after commencing use of the rinse.

While what has been described hereinabove is the preferred embodiment of the invention, it will be understood that formulation of the rinse may be changed without departing from the spirit and scope of the invention. Furthermore, the foregoing description is for the purpose of it illustration only, and not for the purpose of limitation, and the invention is defined by the claims.

For example, the applicant's novel pharmaceutical preparation may be formulated into a lozenge form that may be placed in the mouth and held against an apthous lesion, or gel form that may be formulated into a gel form that may be topically applied to apthous lesions.

What is claimed is:

1. A pharmaceutical preparation for treating apthous ulcers in the mouth consisting essentially of ethyl alcohol in a concentration of about 50% weight per unit volume.

2. The invention in accordance with claim 1 further comprising lidocaine or a pharmaceutically acceptable salt thereof in a concentration of about 2% weight per unit volume.

3. The invention in accordance with claim 1 wherein the pharmaceutical preparation is in the form of a liquid rinse and the mouth is rinsed with the liquid rinse a plurality of times every day until the ulcer is healed.

4. The invention in accordance with claim 1 wherein the pharmaceutical preparation is in the form of a gel that is topically applied to apthous ulcers a plurality of times every day until the ulcer is healed.

5. The invention in accordance with claim 1 wherein the pharmaceutical preparation is in the form of a lozenge that is placed against an apthous ulcer a plurality of times every day until the ulcer is healed.

6. The invention in accordance with claim 2 wherein the pharmaceutical preparation is in the form of a liquid rinse and the mouth is rinsed with the liquid rinse a plurality of times every day until the ulcer is healed.

7. The invention in accordance with claim 2 wherein the pharmaceutical preparation is in the form of a gel that is topically applied to apthous ulcers a plurality of times every day until the ulcer is healed.

8. The invention in accordance with claim 2 wherein the pharmaceutical preparation is in the form of a lozenge that is placed against an apthous ulcer a plurality of times every day until the ulcer is healed.

9. A method for treating apthous ulcers in the mouth by applying a pharmaceutical preparation consisting essentially of ethyl alcohol in a concentration of about 50% weight per unit volume.

10. The method in accordance with claim 9 wherein the pharmaceutical preparation further comprises lidocaine or a pharmaceutically acceptable salt thereof in a concentration of about 2% weight per unit volume

11. The method in accordance with claim 9 wherein the pharmaceutical preparation is in the form of a liquid rinse and the mouth is rinsed with the liquid rinse a plurality of times every day until the ulcer is healed.

12. The method in accordance with claim 9 wherein the pharmaceutical preparation is in the form of a gel that is topically applied to apthous ulcers a plurality of times every day until the ulcer is healed.

13. The method in accordance with claim 9 wherein the pharmaceutical preparation is in the form of a lozenge that is placed against an apthous ulcer a plurality of times every day until the ulcer is healed.

14. The method in accordance with claim 10 wherein the pharmaceutical preparation is in the form of a liquid rinse and the mouth is rinsed with the liquid rinse a plurality of times every day until the ulcer is healed.

15. The method in accordance with claim 10 wherein the pharmaceutical preparation is in the form of a gel that is topically applied to apthous ulcers a plurality of times every day until the ulcer is healed.

16. The method in accordance with claim 10 wherein the pharmaceutical preparation is in the form of a lozenge that is placed against an apthous ulcer a plurality of times every day until the ulcer is healed.
